(19) United States
(12) Patent Application Publication
(10) Pub. No.: US 2007/0128722 A1
(43) Pub. Date: Jun. 7, 2007

(54) Human mesenchymal stem cells and culturing methods thereof

(75) Inventors: Pei-Ju Lin, Taipei City (TW); Cheng-Yi Wu, Taiping City (TW); Hui-Ti Lin, Hsintien City (TW); Wannhsin Chen, Hsinchu City (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(73) Assignee: Industrial Technology Research Institute

(21) Appl. No.: 11/293,991

(22) Filed: Dec. 5, 2005

(51) Int. Cl. C12N 5/08

(52) U.S. Cl. 435/366; 435/372

(57) ABSTRACT

Provided herein are methods of proliferating human mesenchymal stem cells obtained from human cord blood and/or human bone marrow aspirates comprising culturing the human mesenchymal stem cells in an environment containing extracellular matrix isolated form human fibroblasts.

### BACKGROUND

1. Field of Invention

The present invention relates to adult human mesenchymal stem cells obtained from human cord blood and/or human bone marrow aspirates and their methods of culturing.

2. Description of Related Art

Stem cells have the potential of developing into many different cell types in the body. Theoretically, stem cells can divide without limit to replenish other cells. When a stem cell divides, each new cell has the potential to either remain as a stem cell or become another type of cell with a more specialized function, such as a muscle cell, a red blood cell, or a brain cell. Stem cells are often classified as totipotent, pluripotent, and multipotent. A totipotent stem cell has differentiation potential which is total: it gives rise to all the different types of cells in the body, including the germ cells. A fertilized egg cell is an example of a totipotent stem cell. Pluripotent stem cells can give rise to any type of cell in the body except those needed to develop a fetus. Multipotent stem cells can give rise to two or more different cell types but only within a given organ or tissue type.

The main sources of stem cells are the embryonic stem cells and adult stem cells. Embryonic stem cells are derived from embryos. For research purposes, embryonic stem cells are obtained from embryos that have developed from eggs that have been fertilized in vitro (such as at an in vitro fertilization clinic) and then donated for research purposes with informed consent of the donors. The embryos are typically obtained at four or five days old when they are a hollow microscopic ball of cells called the blastocyst. The blastocyst includes three structures: the trophoblast, which is the layer of cells that surrounds the blastocyst; the blastocoel, which is the hollow cavity inside the blastocyst; and the inner cell mass, which is a group of approximately 40 to 150 cells at one end of the blastocoel. The embryonic stem cells are obtained by isolating the inner cell mass and growing them in vitro. The inner cell mass is usually grown on a layer of feeder cells, such as embryonic fibroblasts that serve as an adherent layer for the inner cell mass and as a source of nutrients. Embryonic stem cells are pluripotent and can become all cell types of the body.

An adult stem cell, or a somatic stem cell, is multipotent and an undifferentiated cell found among differentiated cells in a tissue or organ. An adult stem cell can renew itself and can differentiate into specialized cell types of the tissue or organ. They are believed to reside in a specific area of each tissue where they may remain quiescent (non-dividing) for many years until they are activated by disease or tissue injury. Adult stem cells are present in very small numbers in each tissue and have been found in various tissues and organ, including the brain, bone marrow, peripheral blood, blood vessels, skeletal muscle, skin, umbilical cord, adipose tissue, and liver. The plastic-adherent cells isolated from bone marrow and other sources are known as multipotent mesenchymal stromal cells or called mesenchymal stem cells (MSCs) when they meet specified stem cell criteria (Horwitz et al., Cytotherapy 7(5): 393-395, 2005).

Stem cells have gained considerable interest as a treatment for a myriad of diseases, conditions, and disabilities because they provide a renewable source of cells and tissues. An advantage of adult stem cells is that the patient's own cells may be expanded in culture and reintroduced into the patient. The use of the patient's own adult stem cells would prevent rejection of the cells by the immune system without having to use immunosuppressive drugs.

The use of embryonic stem cells in the treatment of diseases is controversial because of its implications on life. In contrast, adult stem cells pose no ethical dilemma, but they are generally limited to differentiating into cell types of their tissue of origin, although, some evidence do suggest that adult stem cell may differentiate into other cell types. For example, hematopoietic stem cells (HSCs) or blood-forming stem cells that found in bone marrow, may differentiate into brain cells such as neurons, oligodendrocytes, and astrocytes (Hao et al., H. Hematother. Stem Cell Res. 12:23-32, 2003; Zhao et al., PNAS 100:2426-2431, 2003; Bonilla et al., Eur. J. Neurosci. 15:575-582, 2002), skeletal muscle cells (Ferrari et al., Science 279:1528-1530, 1998; Gussoni et al., Nature 401:390-394, 1999), cardiac muscle cells (Jackson et al., J. Clin. Invest. 107:1395-1402, 2001), and liver cells (Lagasse et al., Nat. Med. 6:1229-1234, 2000). Bone marrow stromal cells may differentiate into cardiac muscle cells and skeletal muscle cells (Galmiche et al., Blood 82:66-76, 1993; Wakitani et al., Muscle Nerve 18:1417-1426, 1995), while brain stem cells may differentiate into blood cells (Bjornson et al., Science 283:534-547, 1999) and skeletal muscle cells (Galli et al., Nat. Neurosci. 3:986-991, 2000).

Due to the reason that adult stem cells are rare in adult tissues and it is difficult to expand their numbers in cell culture, methods of proliferating adult stem cells in culture are sought, in hope that sufficient number of adult stem cells may be obtained for further practical clinical purpose. JP Patent Publication No.: 2003-052360 and a published paper (Matsubara et al., Biochem. Biophys. Res. Comm. 313:503-508, 2004) disclosed a method of culturing mesenchymal stem cells in tissue culture dishes coated with basement membrane-like extracellular matrix (ECM), which was produced by primary mouse endothelial cells (PYS-2 cells) or by bovine corneal endothelial cells. It is found that the stem cells expanded on ECM-coated culture dishes, but not on un-coated plastic culture dishes, retained the multi-lineage differentiation potential throughout many mitotic division. Unfortunately, in clinical application, the use of stem cells that were cultured in the presence of ECM originated from mouse or bovine will put the potential recipient of the stem cells in a disadvantageous position of having xenogenetic contamination or developing heterlogous rejection. In this respect, there exist in this art a need of an improved method of proliferating human mesenchymal stem cells that are free of xenogenetic contamination and/or heterlogous rejection in the recipients of the stem cells.

### SUMMARY

The present invention provides methods of proliferating human mesenchymal stem cells. Particularly, methods of culturing human mesenchymal stem cells obtained from human cord blood and/or human bone marrow aspirates in an environment containing extracellular matrix (ECM) isolated form human fibroblasts.

One aspect of the invention provides a method for proliferating a human mesenchymal stem cell comprising: obtaining post-partum umbilical cord blood; preparing a single-cell suspension of mononuclear cells (MNCs) from the cord blood; obtaining the mesenchymal stem cells; culturing the mesenchymal stem cells in an environment containing ECM isolated from human fibroblasts. At least 76% of the cultured stem cells remain undifferentiated and multipotent for at least 9 passages (P9 or P3+6, meaning cells that were passage for 3 times and then continued to culture on ECM from P4 for another 6 passages).

Another aspect of the invention provides a method for proliferating a human mesenchymal stem cell comprising: preparing a single-cell suspension of MNCs from human bone marrow aspirates; obtaining human mesenchymal stem cells; and culturing the stem cells in an environment containing ECM isolated from human fibroblasts. At least 76% of the cultured stem cells remain undifferentiated and multipotent for at least 8 passages (P8 or P2+6, meaning cells were passage for 2 times and then continued to culture on ECM from P3 for another 6 passages).

A further aspect of the present invention provides a system for supporting cell-growth, maintaining undifferentiated state or enhancing differentiation capability of human mesenchymal stem cells, comprising: a substrate covered with ECM isolated from human fibroblasts; and an isolated human mesenchymal stem cells; wherein the cultured stem cells having the following characteristics:

a. At least 76% of the cultured stem cells remain undifferentiated for at least 8 passages (P8 or P2+6) or 9 passages (P9 or P3+6); and

b. positive for at least one of the cell markers selected from the group consisting of CD29, CD44, CD90/Thy-1, CD105, CD166, stro-1, SH2, SH3, SH4 and vimentin, and negative for at least one of the cell markers selected from the group consisting of CD31, CD34 and CD45.

Another aspect of the invention provides an isolated, population of multipotent human mesenchymal stem cells cultured by the method of this invention characterized in having certain characteristics, including cell markers. Another aspect of the invention provides cryopreserved mesenchymal stem cells cultured by the method of this invention and characterized in having certain characteristics, including cell markers.

Other aspects of the invention provide. a composition comprising a human mesenchymal stem cell and a pharmaceutical composition comprising a human mesenchymal stem cell. The invention also provides a method of treating a patient comprising administering to the patient an effective amount of a human mesenchymal stem cell.

It is to be understood that both the foregoing general description and the following detailed description are by examples, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 shows the proliferation by cell numbers of BMSC (FIG. 1A) or CB-MSC (FIG. 1B) cultured according to one preferred embodiment of the invention;

FIG. 2 shows the cell-length measurements of BMSC (FIG. 2A) or CB-MSC (FIG. 2B) cultured according to one preferred embodiment of the invention;

FIG. 3 shows the cell surface marker expression in BMSC (panel A) or CB-MSC (panel B) cultured according to one preferred embodiment of the invention and analyzed by flow cytometry; and

FIG. 4 shows the chondrogenic (panel A), osteogenic (panel B), and adipogenic (panel C) gene expression of BMSCs cultured according to one preferred embodiment of the invention.

### DESCRIPTION OF THE PREFERRED EMBODIMENTS

The embodiments described and the terminology used herein are for the purpose of describing exemplary embodiments only, and are not intended to be limiting. The scope of the present invention is intended to encompass additional embodiments not specifically described herein, but that would be apparent to one skilled in the art upon reading the present disclosure and practicing the invention.

As used herein, the term “stem cell” refers to a master cell that can reproduce indefinitely to form the specialized cells of tissues and organs. A stem cell can divide to produce two daughter stem cells, or one daughter stem cell and one progenitor cell, which then proliferates into the tissue's mature, fully formed cells. As used herein, the term “stem cell” includes multipotent and pluripotent stem cells.

As used herein, the term “pluripotent cell” refers to a cell that has complete differentiation versatility, i.e., the capacity to grow into any of the mammalian body's cell types, except those needed to develop a fetus. A pluripotent cell can be self-renewing, and can remain dormant or quiescent within a tissue. As used herein, the term “multipotent cell” refers to a cell that has the capacity to grow into two or more different cell types of the mammalian body within a given tissue or organ.

As used herein, the term “ECM” refers to a particulate a cellular matrix composed of extracellular and cellular matrices isolated from human fibroblasts. In one preferred embodiment, the human fibroblasts are human foreskin fibroblasts, however, other types of fibroblasts may also be used. ECM may be prepared by methods known in the art, such as Jordana et al. Eur. Respir. J. 7: 2106, 1994; and U.S. Pat. No. 4,816,561. In general, ECM is prepared by lysing the fibroblast cells with an alkali solution and followed by rinsing with sufficient amount of buffer solution, so that only the cytoskeleton and ECM proteins such as collagen, elastins, fibrillin, fibronectin, and laminin and glycans such as proteoglycans and glycosaminoglycans (GAGs) are preserved after washing. The ECM thus prepared is used as a scaffold for the stem cells of this invention to grown and/or proliferate on.

As used herein, the number of passage of a cell in the culture is expressed as a capital letter “P” followed by “a numerical number”. For example, “P6” refers to cells that have been passage for a total of 6 times. Another expression that are used throughout the specification is “P(N1)+(N2)”, wherein N1 represents the passage number of a cells before being cultured on the ECM, whereas N2 represents the passage number of a cells after being cultured on the ECM,. For example, “P2+4” refers to cells that have been passage for 2 times and then continue to culture on ECM for another 4 passages.

The present invention thus provides a method of proliferating adult human mesenchymal stem cells comprising the steps of: obtaining a single-cell suspension of MNCs from post-partum umbilical cord blood and/or human bone marrow aspirates; obtaining the mesenchymal stem cells; and culturing the stem cells in the presence of ECM isolated form human fibroblasts.

The post-partum umbilical cord may be obtained, for example, with informed consent from a woman underwent caesarian procedure or normal birth. The bone marrow aspirates may obtain from a suitable donor or any commercial source. The cord blood may be drawn and collected by a syringe. A single-cell suspension of MNCs may be prepared by centrifugation according to Boyum A., Scand. J. Clin. Lab. Invest. 21 Suppl. 97 (Paper IV): 77-89, 1968. The obtained mesenchymal stem cells are then cultured in culture dishes pre-covered with ECM prepared by the procedure described above. The culture medium comprising standard medium, such as α-MEM (Gibco) and 10% fetal bovine serum (FBS), and may be optionally supplemented with growth factors such as fibroblast growth factors (FGFs) as appropriate. Mesenchymal stem cells may be obtained by continued culture of the mesenchymal stem cells in the culture medium for at least 8 to 9 passages on the ECM.

A mesenchymal stem cell may be characterized by its cell markers. A variety of cell markers are known. See e.g., Stem Cells: Scientific Progress and Future Research Directions. Appendix E. II. Markers Commonly Used To Identify Stem Cells and To Characterize Differentiated Cell Types. Department of Health and Human Services. June 2001. http://www.nih.gov/news/stemcell/scireport.htm. Cell markers may be detected by methods known in the art, such as by immunochemistry or flow cytometry. Flow cytometry allows the rapid measurement of light scatter and fluorescence emission produced by suitably illuminated cells or particles. The cells or particles produce signals when they pass individually through a beam of light. Each particle or cell is measured separately and the output represents cumulative individual cytometric characteristics. Antibodies specific to a cell marker may be labeled with a fluorochrome so that it may be detected by the flow cytometer. See, eg., Ormerod (ed.), Flow Cytometry: A Practical Approach, Oxford Univ. Press, 1997.

In an embodiment of the invention, a human mesenchymal stem cell cultured according to the method of this invention expresses at least one of the following cell markers: CD29, CD44, CD90/Thy-1, CD105, CD166, stro-1, SH2, SH3, SH4 and vimentin. In a further embodiment, a human mesenchymal stem cell is negative for at least one of the following cell markers: CD31, CD34 and CD45.

The present invention also embodies a homogeneous population of mesenchymal stem cells. As used herein, “homogeneous population” refers to a population of cells exhibiting substantially the same phenotype, such as that determined by cell markers. An isolated population prepared according to the method of this invention may comprise at least about 76% of substantially the same cells, or at least about 83%, 84%, 88%, 89%, 90%, 91%, 93%, 95%, 96%, 97%, or 98% of substantially the same cells. Specifically, a population of mesenchymal stem cells isolated form human bone marrow aspirates (BMSC) comprises at least 88% of substantially the same cells after 4 passages (or P2+2); at least 84% of substantially the same cells after 6 passages (or P2+4); and at least 76% of substantially the same cells after 8 passages (P2+6). A homogeneous population of mesenchymal stem cells isolated form human cord blood (CB-MSC) comprises at least 83% of substantially the same cells after 5 passages (P3+2); at least 93% of substantially the same cells after 7 passages (P3+4); and at least 76% of substantially the same cells after 9 passages (P3+6).

In an embodiment of the present invention, human mesenchymal stem cells are proliferating in a system that is capable of supporting the growth of the stem cells, maintaining the undifferentiated states of the stem cells or enhancing differentiating capability of the stem cells, comprising:

a substrate covered with ECM isolated from human fibroblasts; and

an isolated human mesenchymal stem cells from human cord blood or human bone marrow aspirates;

wherein the cultured mesenchymal stem cells having at least one of the following characteristics:

at least 76% of the stem cells remains undifferentiated for at least 8 passages (P8, or P2+6) or 9 passages (P9, or P3+6);
the stem cells is characterized in being positive for at least one of the cell markers CD29, CD44, CD90/Thy-1, CD105, CD166, stro-1, SH2, SH3, SH4 and vimentin; and being negative for cell markers CD34 and CD45.

The stem cells in culture may be detected by their ability to differentiate into different cell types. For example, the cultured cells may be tested for their ability to undergo adipogenic, and/or osteochondrogenic differentiation. Adipocytes are connective tissue cells responsible for the synthesis and storage of fat, while chondrocytes and osteoblasts are the primary cells responsible for bone formation and are thought to originate from osteoprogenitor cells within skeletal tissues. In general, differentiation of mesenchymal stem cells were induced and detected according to the method described by Matsubara et al., Biochem. Biophys. Res. Comm. 313:503-508, 2004.

Specifically, adipogenic differentiation is induced by culturing the mesenchymal stem cells in an adipogenic differentiation medium containing DMEM-LG supplemented with 10% FBS, 1 μM dexamethasone, 10 μM insulin, 0.5 mM isobutyl- methylxanthine, and 200 μM indomethacin. Adipogenic differentiation may be detected by testing for the presence of adipogenic transcription factors PPARy2 (peroxisome proliferator activator receptor gamma) by RT-PCR.

Osteogenic differentiation is initiated by culturing the mesenchymal stem cells in an osteogenic differentiation medium containing DMEM-LG supplemented with 10% FBS, 10 mM glycerolphosphate (Sigma), 50 μM ascorbate-2-phosphate, and 0.1 μM dexamethasone (Sigma). Osteogenic differentiation may be detected by testing for the presence of osteogenic markers, which include, but are not limited to, osteopontin (OP), osteocalcin (OC), osteonectin (ON) by RT-PCR.

Chondrogenic differentiation is initiated by culturing the mesenchymal stem cells in a chondrogenic differentiation medium containing DMEM-LG supplemented with 1% FBS, 10 ng/ml TGF-β1 (R&D), 50 nM ascorbate-2-phosphate (Sigma), and 6.25 μg/ml insulin (Sigma). Chondrogenic differentiation is detected by testing for the presence of chondrogenic markers such as type X collagen and type II collagen by RT-PCR.

The present invention further provides a composition comprising a mesenchymal stem cell of the invention. The present invention also provides a pharmaceutical composition comprising a mesenchymal stem cell of the invention. The mesenchymal stem cell of the invention or formulations thereof may be administered by any conventional method including parenteral (e.g. subcutaneous or intramuscular) injection or intravenous infusion. The treatment may consist of a single dose or a plurality of doses over a period of time. The pharmaceutical composition may comprise one or more acceptable carriers. The carrier(s) must be “acceptable” in the sense of being compatible with the mesenchymal stem cells and not deleterious to the recipients thereof. Typically, the carriers may be water or saline which will be sterile and pyrogen free.

The mesenchymal stem cells of the invention may also be cryopreserved. The cells may be cryopreserved in a solution comprising, for example, dimethyl sulfoxide at a final concentration not exceeding 10%. The cells may also be cryopreserved in a solution comprising dimethyl sulfoxide and/or dextran. Other methods of cryopreserving cells are known in the art.

The present invention provides a method of treating a patient, which comprises administering to the patient a therapeutically effective amount of the mesenchymal stem cell of the invention. “Therapeutically effective amount” as used herein, refers to that amount of mesenchymal stem cell that is sufficient to reduce the symptoms of the disorder, or an amount that is sufficient to maintain or increase in the patient the number of cells derived from the mesenchymal stem cell. A patient is hereby defined as any person in need of treatment with a mesenchymal stem cell. The mesenchymal stem cells of the invention may be used in the treatment of any kind of injury due to trauma where tissues need to be replaced or regenerated. Examples of such trauma-related conditions include central nervous system (CNS) injuries, including injuries to the brain, spinal cord, or tissue surrounding the CNS injuries to the peripheral nervous system (PNS), or injuries to any other part of the body. Such trauma may be caused by accident, or may be a normal or abnormal outcome of a medical procedure such as surgery or angioplasty. In specific embodiments, the cells may be used in autologous or heterologous tissue replacement or regeneration therapies or protocols, including, but not limited to treatment of corneal epithelial defects, cartilage repair, facial dermabrasion, mucosal membranes, tympanic membranes, intestinal linings, neurological structures (e.g., retina, auditory neurons in basilar membrane, olfactory neurons in olfactory epithelium), burn and wound repair for traumatic injuries of the skin, or for reconstruction of other damaged or diseased organs or tissues. Injuries may be due to specific conditions and disorders including, but not limited to, myocardial infarction, seizure disorder, multiple sclerosis, stroke, hypotension, cardiac arrest, ischemia, inflammation, age-related loss of cognitive function, radiation damage, cerebral palsy, neurodegenerative disease, Alzheimer's disease, Parkinson's disease, Leigh disease, AIDS dementia, memory loss, amyotrophic lateral sclerosis (ALS), ischemic renal disease, brain or spinal cord trauma, heart-lung bypass, glaucoma, retinal ischemia, retinal trauma, inborn errors of metabolism, adrenoleukodystrophy, cystic fibrosis, glycogen storage disease, hypothyroidism, sickle cell anemia, Pearson syndrome, Pompe's disease, phenylketonuria (PKU), porphyrias, maple syrup urine disease, homocystinuria, mucoplysaccharide nosis, chronic granulomatous disease and tyrosinemia, cancer, tumors or other pathological or neoplastic conditions.

The mesenchymal stem cell used in the treatment may also contain a nucleic acid vector or biological vector in an amount sufficient to direct the expression of a desired gene(s) in a patient. The construction and expression of conventional recombinant nucleic acid vectors is well known in the art and includes those techniques contained in Sambrook et al., Molecular Cloning: A Laboratory Manual, Vols 1-3 (2nd ed. 1989), Cold Spring Harbor Laboratory Press. Such nucleic acid vectors may be contained in a biological vector such as viruses and bacteria, preferably in a non-pathogenic or attenuated microorganism, including attenuated viruses, bacteria, parasites, and virus-like particles.

The nucleic acid vector or biological vector may be introduced into the cells by an ex vivo gene therapy protocol, which comprises excising cells or tissues from a patient, introducing the nucleic acid vector or biological vector into the excised cells or tissues, and re-implanting the cells or tissues into the patient (see, for example, Culver et al., Hum. Gene Ther. 1:399-410, 1990; Kasid et al., Proc. Natl. Acad. Sci. U.S.A. 87:473-477, 1990). The nucleic acid vector or biological vector may be introduced into excised cells or tissues by, for example, calcium phosphate-mediated transfection. Other techniques for introducing nucleic acid vectors into host cells, such as electroporation (Neumann et al., EMBO J. 1:841-845, 1982), may also be used.

The cells of the invention may also be co-administered with other agents, such as other cell types, growth factors, and antibiotics. Other agents may be determined by those of ordinary skill in the art.

Other than in the operating examples, or where otherwise indicated, all numbers expressing quantities of ingredients, reaction conditions, and so forth used in this application are to be understood as being modified in all instances by the term “about.” Accordingly, unless the contrary is indicated, the numerical parameters set forth in this application are approximations that may vary depending upon the desired properties sought to obtain by the present invention. At the very least, and not as an attempt to limit the application of the doctrine of equivalents to the scope of the claims, each numerical parameter should be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements.

It must be noted that as used herein and in the appended claims, the singular forms “a”, “an”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice of the present invention, exemplary methods and materials are described for illustrative purposes.

All publications mentioned in this application are incorporated by reference to disclose and describe the methods and/or materials in connection with which the publications are cited. Additionally, the publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Methods, techniques, and/or protocols (collectively “methods”) that can be used in the practice of the invention are not limited to the particular examples of these procedures cited throughout the specification but embrace any procedure known in the art for the same purpose. Furthermore, although some methods may be described in a particular context in the specification, their use in the instant invention is not limited to that context.

### EXAMPLES

The following Examples are provided to illustrate certain aspects of the present invention and to aid those of skill in the art in practicing this invention. These Examples are in no way to be considered to limit the scope of the invention in any manner.

### Example 1

Passages of Human Mesenchymal Stem Cells in Culture Dishes Covered with Extracellular Matrix

1.1 Isolation and Culture of Human Mesenchymal Stem Cells

1.1.1 Isolation and Culture of Human Mesenchymal Stem Cells from Human Bone Marrow (BMSC)

Frozen human bone marrow aspirates (obtained from Cambrex Inc. Lot No.: 0313557) was thawed in a water bath at 37° C., and then transferred to a centrifuged tube, about 50 ml culture medium were added dropwisely in a period of 10-15 min. The cells were pelleted by centrifugation at a speed of 200×g for 15 min, then re-suspended in 15 ml culture medium. Repeat the centrifugation step once, then the obtained mesenchymal stem cells (BMSC) were counted and plated in culture dishes in α-MEM medium (obtained from Gibco, Cat. No.: 12571-063) supplemented with 20% fetal bovine serum. Cell cultures were maintained at 37° C. and 5% CO₂ and in a water-saturated atmosphere for 7-10 days. Non-adherent cells were removed by a few washes with culture medium and the adherent cells were further cultured until 80% confluent. The cells were then harvested with 0.25% trypsin and 1 mM EDTA (Gibco) for 5 minutes at 37° C., and re-plated at the density of 50 cells/cm² in a 180-cm² culture flask (Falcon). After 8 days, the cells from the second passage were harvested with trypsin/EDTA, suspended at 1×10⁶cells/ml in 10% DMSO and 90% FBS, and stored in 1-ml aliquots in liquid nitrogen until further use.

1.1.2 Isolation of Human Mesenchymal Stem Cells from Human Cord Blood (CB-MSC)

Fresh umbilical cord was obtained from post-partum woman and the cord blood was drawn by a syringe and centrifuged at a speed of 2000 rpm for 20 min. Small aliquots of the upper plasma fraction were taken out and tested for either HBV or HIV. The upper plasma fraction was decanted, and the buffy coat in the middle layer was taken out carefully and transferred to another centrifuged tube, and mixed with equal volume of phosphate-buffered saline supplemented with 2 mM EDTA (D-PBS/2 mM EDTA). The MNCs were separated by use of a Ficoll (obtained from Amersham Biosciences, Cat. No.: 17-1440-02) density gradient at a speed of 2000 rpm for 40 min, then washed once by D-PBS/2 mM EDTA, and pelleted again by centrifugation at a speed of 1000 rpm for 5 min. The washing was repeated several times or further treated with lysis buffer, which is composed of 150 mM ammonium chloride and 10 mM sodium bicarbonate, until no more erythrocytes could be found. The harvested mesenchymal stem cells (CB-MSC) were re-suspended in α-MEM medium and mixed well with freezing medium which is composed of 5% DMSO, 30% FBS, and 65% α-MEM medium and kept frozen in liquid nitrogen at −80° C. until further use.

1.2 Preparation of Culture Dishes Covered With Extracellular Matrix

1.2.1 Culture Dishes covered with Stematrix

Human foreskin fibroblasts (either obtained from Taiwan Animal Technology Institute, Lot No.: 881122-02-F (HSF) or from American Type Cell culture, ATCC® No. SCRC-1041™, cell line HFF-1, (HFF)) were treated with 10 μg/ml mitomycin C at 37° C. for 3 hrs, and were then seeded at 6×10⁵ cells on 3-cm culture dishes pre-coated with 0.1% gelatin. For preparation of ECM, cells were washed with PBS twice, then lysed with 0.05N NaOH for a period of 1-2 min and rinsed with PBS buffer three times. The extracellular matrix of human foreskin fibroblasts thus prepared is termed Stematrix, and can be used fresh or stored away for future use in PBS at 4° C. for up to 6 months.

1.2.2 Culture Dishes covered with Human Placenta ECM

Human placenta ECM (obtained from BD Pharmingen, cat. No.: 354237) was thawed at 4° C., and diluted with ice-cold α-MEM medium until a final concentration of 25 μg/ml was reached. The diluted human placenta ECM was then used to coat the culture dishes, 1 ml per one 3-cm dish. The coated dishes were let stand undisturbed for 2 hrs at room temperature, then washed twice with α-MEM medium until further use.

1.2.3 Culture Dishes coated with Matrigel™

Matrigel™ (obtained from BD Pharmingen, cat. No.: 354234) was thawed at 4° C., and diluted with ice-cold α-MEM medium, then was used to coat the culture dishes, 1 ml per one 3-cm dish. The coated dishes were let stand undisturbed for 2 hrs at room temperature, then washed twice with α-MEM medium until further uses.

1.2.4 Culture Dishes covered with Mouse ECM

Mouse embryonic fibroblasts were isolated from 13-days-old 129 sv×129 sv strain mouse fetus according to a protocol of Robertson (Robertson E. J. (1987) Embryo-derived stem cell line. Chapter 4 in “Teratocarcinoma and Embryonic Stem Cells: A Practical approach”, IRL Press, Oxford, Washington D.C., p77-78.). The isolated fibroblasts were first treated with 10 μg/ml mitomycin C at 37° C. for 2.5 hrs, and then were seeded at 7×10⁵ cells on 3-cm culture dishes pre-coated with 0.1% gelatin. For preparation of ECM, cells were washed with PBS twice, then lysed with 0.05N NaOH for a period of 1-2 min and rinsed with PBS buffer three times. The extracellular matrix of mouse embryonic fibroblasts thus obtained is termed mECM, and can be used fresh or stored away for future use in PBS at 4° C. for up to 6 months.

1.2.5 Culture Dishes covered with Bovine ECM

Bovine corneal endothelial cells (obtained from Taiwan Animal Technology Institute, Lot No.: 60044) were seeded at 2×10⁴ cells on 6-cm culture dishes in medium A (DMEM-Ham's F12 (1:1), 10% FBS, and antibiotics (100 u/mi penicillin G and 100 μg/ml streptomycin)) at 37° C. (Matsubara et al., Biochem. Biohys. Res. Comm. 313:503-508, 2004 and Gospodarowicz et al., Proc. Natl. Acad. Sci. USA 77(7): 4494-4098,1980). After confluence, the culture medium was changed to medium B (medium A supplemented with 5% dextran (200,000 Dalton, obtained from Wako, Japan)) and the cells were continued to culture for another 7 days. For preparation of ECM, cells were washed with PBS twice, then lysed with 0.5% Triton X-100 (in PBS) for a period of 30 min and rinsed with PBS buffer three times. The extracellular matrix of bovine corneal endothelial cells thus prepared is termed bECM, and can be used fresh or stored away for future use in PBS at 4° C. for up to 2 months.

1.3 Culture of Human Mesenchymal Stem Cells in Dishes Covered With ECM

Human mesenchymal stem cells, both BMSC or CB-MSC, isolated and cultured according to the procedures described in Example 1.1 were seeded in low density (50 cells/cm²) onto the culture dishes of Example 1.2. Culture medium was replaced every 2-3 days and cell cultures were maintained at 37° C. and 5% CO₂ and in a water-saturated atmosphere for 7 days. After one week in culture, the cells were recovered with 0.25% trypsin-EDTA. After counting, the cells were either analyzed (i.e., measurement of cell-length and/or cell surface markers) or re-plated under the same condition as described above. Both BMSC and CB-MSC may continue to grow for at least 8 to 9 passages.

### Example 2

Characterization of Human Mesenchymal Stem Cells of Example 1.3

2.1 Proliferation of BMSC or CB-MSC of Example 1.3

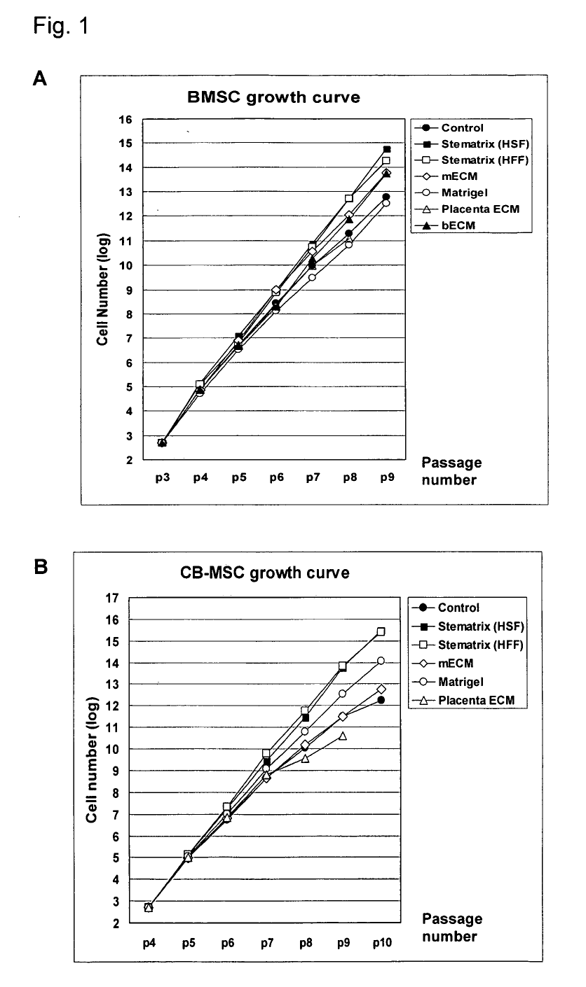

FIG. 1 shows the cell numbers of the BMSC (FIG. 1A) or CB-MSC (FIG. 1B) cultured according to the method described in Example 1.3. It is found that the cell number increased significantly for cells cultured on Stematrix as compared to the control (without ECM coating) and cells plated on other matrices. The result confirms that the proliferation of mesenchymal stem cells is enhanced by the method of this invention. Specifically, the cell number of mesenchymal stem cells cultured in dishes covered with Stematrix (HSF and HFF) is about 30-95 folds for BMSCs at passage 9 (P2+7) (FIG. 1A) and 1,400-1,800 folds for CB-MSCs at passage 10 (P3+7) (FIG. 1B), respectively, compared to the cell numbers of the control cells. Notably, the cell number of BMSCs cultured in dishes covered with Stematrix (HSF and HFF) is about 3-10 folds at passage 9 (P2+7) (FIG. 1A) compared to the cell numbers of BMSCs cultured in dishes covered with bECM, indicating the improvement of the growth of mesenchymal stem cells cultured on Stematrix was superior to some extent than that on bECM in the method described in JP Patent Publication No.: 2003-052360 and Matsubara et al., Biochem. Biophys. Res. Comm. 313:503-508, 2004.

2.2 Undifferentiation of BMSC or CB-MSC of Example 1.3

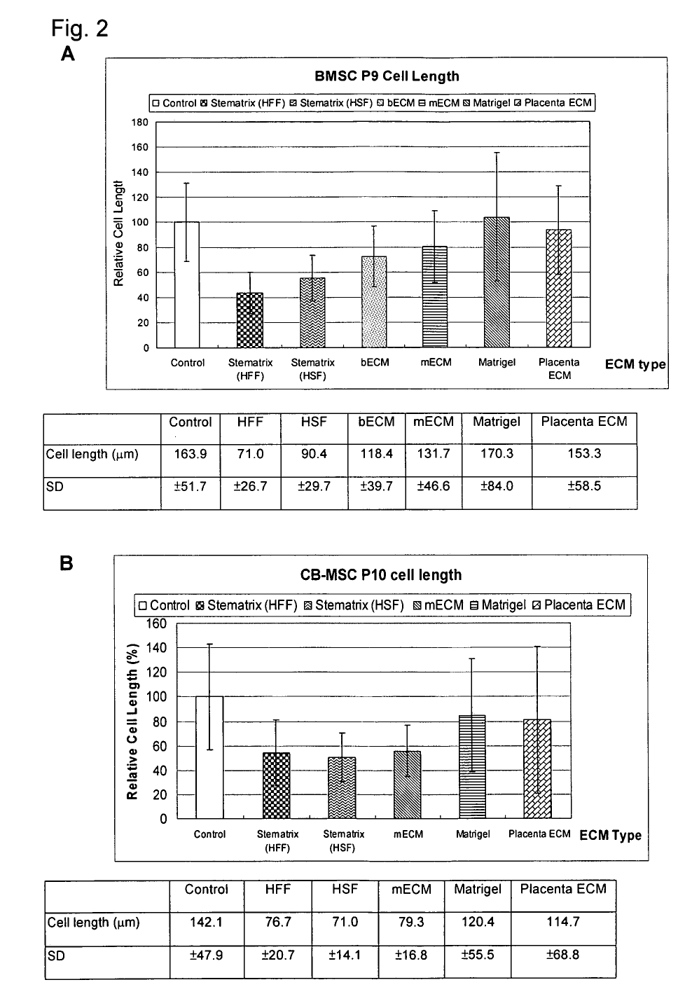

FIG. 2 confirms that most of the BMSC (FIG. 2A) or CB-MSC (FIG. 2B) cultured by the method of this invention possess the morphology of rapidly self-renewing cells (RS cells), which are characterized in having shorter cell-lengths and better capabilities of differentiation (Sekiya et al., Stem Cells, 20: 530-541, 2002), for up to 9 or 10 passages. Cell-length in long axis of the cells was measured by visualizing with an inverted microscope (Nikon, Eclipse TS100) under 100× magnification, and then followed by computer measurement and analysis. Thirty cells in each culture conditions were randomly chosen in the captured images. Specifically, the cell-length of either BMSC at P9 (P2+7) (FIG. is 2A) or CB-MSC at P10 (P3+7) (FIG. 2B) cultured in dishes covered with Stematrix (HSF and HFF) is about 43% to 55% of the cell-length of the control cells (p<0.001 in t tests), i.e., cells plated on regular dishes, indicating that the stem cells maintained in an environment containing ECM isolated from human foreskin fibroblasts are smaller sized cells (potentially RS cells) for up to 9 or 10 passages. More specifically, the cell-length of BMSC cultured on control, Stematrix (HFF), Stematrix (HSF), bECM, mECM, Matrigel™, and Placenta ECM is 163.9±51.7 μm, 71.0±26.7 μcm, 90.5±29.7 μm, 118.4±39.7 μm, 131.7±146.6 μm, 170.3±184.0 μm, and 153.3±158.5 μm, respectively (see FIG. 2A insert table). The cell-length of CB-MSC cultured on control, Stematrix (HFF), Stematrix (HSF), mECM, Matrigel™, and Placenta ECM is 142.1±47.9 μm, 76.7±20.7 μm, 71.0±14.1 μm, 79.3±16. 8 μm, 120.4±55.5 μm, and 114.7±68.8 μm, respectively (see FIG. 2B insert table). Furthermore, the unique benefit of ECM isolated from human foreskin fibroblasts in preventing the mesenchymal stem cells from differentiation is more significant than ECM isolated from other sources, such as ECM that is placenta or mouse embryo origin, see Example 2.3.

2.3 Immunological Characterization of BMSC or CB-MSC of Example 1.3

The human mesenchymal stem cells obtained in Example 1.3 were analyzed for cell markers by flow cytometry and/or immunochemical staining. Briefly, trypsinized cells (cell density) were washed with PBS, stained with phycoerytherin (PE)-conjugated stem cell antibody CD29 or CD90/Thy-1, and incubated on ice for 30 min; or in some cases, incubated on ice for 30 min with stem cell antibody CD31 or CD105, after washing, stained with fluorescein isothiocyanate (FITC)-conjugated goat anti-mouse IgG and incubated on ice for another 30 min. Cells were washed and analyzed on FACScan Flow Cytometer using CellQuest software (Becton Dickinson). See details at http://www.bdbiosciences.com/pharmingen/protocols/Lysed_Whole_Blood_Met hod.shtml.

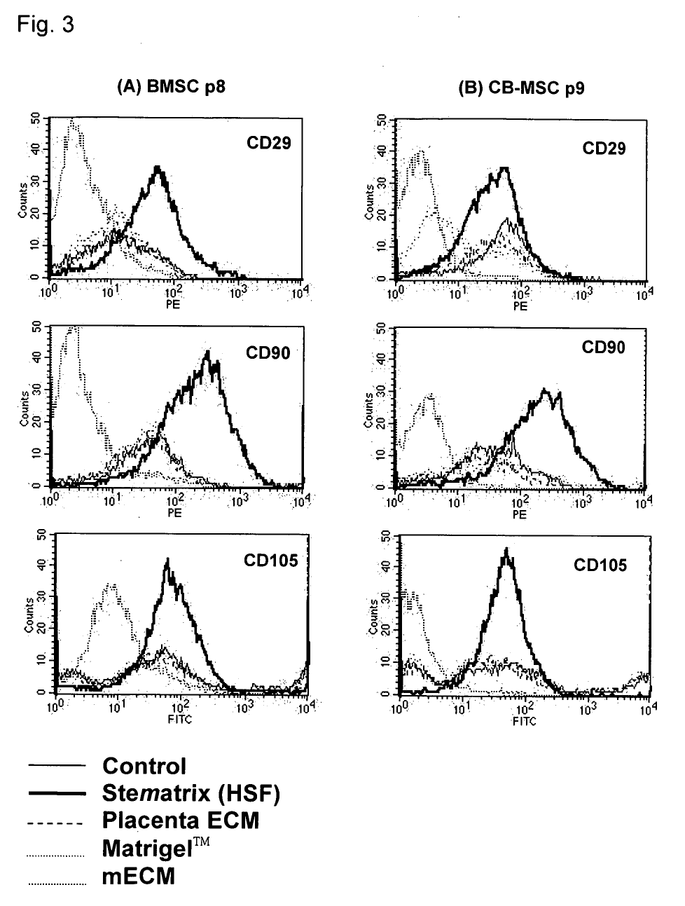

FIG. 3 shows an example of surface marker expression in BMSC (Panel A) and CB-MSC (Panel B) obtained in Example 1.3 by flow cytometry analysis at the seventh or eighth passage, respectively. It is clear that a more significant proportion of stem cells plated on Stematrix expressed CD29, CD90/Thy-1, and CD 105 compared with cells that are plated on plastic dishes (control), Matrigel™, ECM isolated from placenta and mECM.

Table 1 shows a quantified comparison of the cell markers tested on BMSC and CB-MSC at various passages. As the passage number increases, e.g., up to 8 (2+6) or 9 (3+6) passages, the number of staining cells plated in the presence of Matrigel™, ECM isolated from placenta and mECM decreases significantly, from around 90% to 30-40%, or even down to less than 10% in the case of mECM. However, the number of staining cells plated in the presence of Stematrix remains relatively unchanged, and is within 80-90% range.

TABLE 1

| Percentage of | | | | Percentage of | | | |
|---|---|---|---|---|---|---|---|
| BMSC Staining (%) | | | | CB-MSC Staining (%) | | | |
| CD29 | CD90 | CD105 | CD31 | CD29 | CD90 | CD105 | CD31 |

P4 (P2 + 2)

| | | | | | | | | |
|---|---|---|---|---|---|---|---|---|
| Control | 80 | 91 | 85 | 3 | — | — | — | — |
| Stematrix | 90 | 96 | 88 | 4 | — | — | — | — |
| (HSF) | | | | | | | | |
| Placenta | 86 | 97 | 85 | 2 | — | — | — | — |
| ECM | | | | | | | | |
| Matri- | 89 | 93 | 84 | 2 | — | — | — | — |
| gel ™ | | | | | | | | |
| mECM | 89 | 95 | 91 | 1 | — | — | — | — |

P5 (P3 + 2)

| | | | | | | | | |
|---|---|---|---|---|---|---|---|---|
| Control | — | — | — | — | 91 | 94 | 78 | 3 |
| Stematrix | — | — | — | — | 95 | 83 | 89 | 1 |
| (HSF) | | | | | | | | |
| Placenta | — | — | — | — | 90 | 95 | 84 | 3 |
| ECM | | | | | | | | |
| Matri- | — | — | — | — | 94 | 84 | 78 | 4 |
| gel ™ | | | | | | | | |
| mECM | — | — | — | — | 95 | 93 | 93 | 1 |

P6 (P2 + 4)

| | | | | | | | | |
|---|---|---|---|---|---|---|---|---|
| Control | 87 | 93 | 52 | 7 | — | — | — | — |
| Stematrix | 97 | 98 | 84 | 2 | — | — | — | — |
| (HSF) | | | | | | | | |
| Placenta | 78 | 68 | 49 | 9 | — | — | — | — |
| ECM | | | | | | | | |
| Matri- | 89 | 87 | 54 | 7 | — | — | — | — |
| gel ™ | | | | | | | | |
| mECM | 61 | 47 | 25 | 3 | — | — | — | — |

P7 (P3 + 4)

| | | | | | | | | |
|---|---|---|---|---|---|---|---|---|
| Control | — | — | — | — | 77 | 60 | 48 | 14 |
| Stematrix | — | — | — | — | 98 | 93 | 95 | 1 |
| (HSF) | | | | | | | | |
| Placenta | — | — | — | — | 75 | 53 | 46 | 12 |
| ECM | | | | | | | | |
| Matri- | — | — | — | — | 79 | 64 | 50 | 15 |
| gel ™ | | | | | | | | |
| mECM | — | — | — | — | 5 | 2 | 8 | 0.2 |

P8 (P2 + 6)

| | | | | | | | | |
|---|---|---|---|---|---|---|---|---|
| Control | 33 | 67 | 48 | 13 | — | — | — | — |
| Stematrix | 76 | 96 | 84 | 3 | — | — | — | — |
| (HSF) | | | | | | | | |
| Placenta | 34 | 62 | 45 | 13 | — | — | — | — |
| ECM | | | | | | | | |
| Matri- | 25 | 67 | 48 | 14 | — | — | — | — |
| gel ™ | | | | | | | | |
| mECM | 5 | 7 | 16 | 4 | — | — | — | — |

P9 (P3 + 6)

| | | | | | | | | |
|---|---|---|---|---|---|---|---|---|
| Control | — | — | — | — | 84 | 50 | 47 | 13 |
| Stematrix | — | — | — | — | 76 | 91 | 89 | 1 |
| (HSF) | | | | | | | | |
| Placenta | — | — | — | — | 72 | 36 | 46 | 16 |
| ECM | | | | | | | | |
| Matri- | — | — | — | — | 29 | 39 | 43 | 16 |
| gel ™ | | | | | | | | |
| mECM | — | — | — | — | 2 | 1 | 3 | 0.1 |

— undetermined.

2.4 Differentiation of BMSC or CB-MSC of Example 1.3

The human mesenchymal stem cells obtained in Example 1.3 were analyzed for their potentials of chondrogenic, osteogenic, and adipogenic differentiation. The procedures for in vitro differentiation of BMSCs are as those described by Shih et al. Stem Cells 23(7):1012-1020, 2005. RT-PCR analyses of chondrogenic (type X collagen and type 11 collagen), osteogenic (OP and OC), and adipogenic (PPARy2) gene expression are as those described by Matsubara et al., Biochem. Biophys. Res. Comm. 313:503-508, 2004.

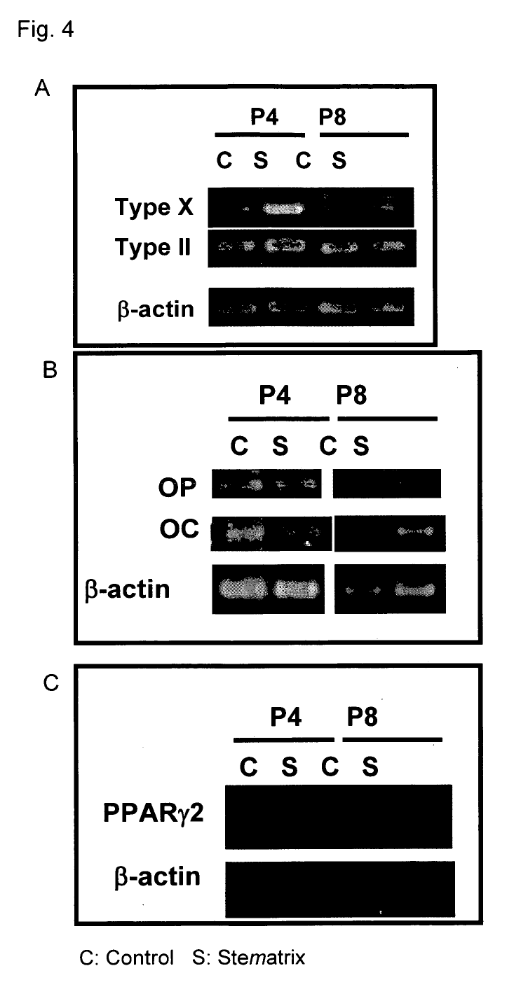

FIG. 4 shows the chondrogenic (panel A), osteogenic (panel B), and adipogenic (panel C) gene expression of BMSCs (P4 (or P2+2) and P8 (or P2+6), respectively) determined by RT-PCR. It is found that the levels of the expressed marker genes in BMSCs that were cultured in dishes covered with Stematrix were either enhanced or at least as the same of the control cells, which were cultured in the absence of ECM. Expression of β-actin is used as an internal control for RT-PCR analysis. Results indicate that mesenchymal stem cells cultured in accordance with the preferred method of this invention remain multipotent, or the ability to differentiate, for at least 8 passages, or 6 passages on the ECM, to be exactly.

The specification is most thoroughly understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with the true scope and spirit of the invention being indicated by the following claims.

What is claimed is:

1. A method for proliferating human mesenchymal stem cells comprising culturing the human mesenchymal stem cells in an environment containing extracellular matrix isolated from human fibroblasts.

2. The method of claim 1, wherein the stem cells is isolated from human cord blood or human bone marrow aspirates.

3. The method of claim 1, wherein at least 76% of the human mesenchymal stem cells remains substantially undifferentiated for at least 8 passages.

4. The method of claim 3, wherein the undifferentiated stem cells is characterized in being positive for at least one of cell markers selected from the group consisting of CD29, CD44, CD90/Thy-1, CD105, CD166, stro-1, SH2, SH3, SH4 and vimentin, and being negative for at least one of the cell markers selected from the group consisting of CD31, CD34 and CD45.

5. The method of claim 3, wherein the undifferentiated stem cells are multipotent.

6. The method of claim 1, wherein the human fibroblasts are a primarily isolated cells or an immortalized cell line.

7. The method of claim 1, further comprising adding a fibroblast growth factor to the culture medium.

8. The method of claim 1, wherein the extracellular matrix is prepared by culturing human fibroblasts, lysing the fibroblasts with alkali solution, and then washing what remains after lysing.

9. An isolated, homogenous population of multipotent human mesenchymal stem cells obtained by the method of claim 1.

10. A method for proliferating human mesenchymal stem cells comprising:
obtaining cord blood from a post-partum umbilical cord;
preparing a single-cell suspension of mononuclear cells from the cord blood;
obtaining human mesenchymal stem cells; and
culturing the stem cells in an environment containing extracellular matrix isolated from human fibroblasts.

11. The method of claim 10, wherein at least 76% of the stem cells remains substantially undifferentiated for at least 9 passages.

12. The method of claim 11, wherein the undifferentiated stem cells is characterized in being positive for at least one of the cell markers selected from the group consisting of CD29, CD44, CD90/Thy-1, CD105, CD166, stro-1, SH2, SH3, SH4 and vimentin, and being negative for at least one of the cell markers selected from the group consisting of CD 31, CD34 and CD45.

13. The method of claim 10, wherein the undifferentiated stem cells are multipotent.

14. The method of claim 10, wherein the human fibroblasts are a primarily isolated cells or an immortalized cell line.

15. The method of claim 10, further comprising adding a fibroblast growth factor to the culture medium.

16. The method of claim 10, wherein the extracellular matrix is prepared by culturing human fibroblasts, lysing the fibroblasts with alkali solution, and then washing what remains after lysing.

17. An isolated, homogenous population of multipotent human mesenchymal stem cells obtained by the method of claim 10.

18. A method for proliferating human mesenchymal stem cells comprising:
Obtaining human bone marrow aspirates;
preparing a single-cell suspension of mononuclear cells from the human bone marrow aspirates;
obtaining human mesenchymal stem cells; and
culturing the stem cells in an environment containing extracellular matrix isolated from human fibroblasts.

19. The method of claim 18, wherein at least 76% of the stem cells remains substantially undifferentiated for at least 9 passages.

20. The method of claim 19, wherein the undifferentiated stem cells is characterized in being positive for at least one of the cell markers selected from the group consisting of CD29, CD44, CD90/Thy-1, CD105, CD166, stro-1, SH2, SH3, SH4 and vimentin, and being negative for at least one of the cell markers selected from the group consisting of CD31, CD34 and CD45.

21. The method of claim 18, wherein the undifferentiated stem cells are multipotent.

22. The method of claim 18, wherein the human fibroblasts are a primarily isolated cells or an immortalized cell line.

23. The method of claim 18, further comprising adding a fibroblast growth factor to the culture medium.

24. The method of claim 18, wherein the ECM is prepared by culturing human fibroblasts, lysing the fibroblasts with alkali solution, and then washing what remains after lysing.

25. An isolated, homogenous population of multipotent human mesenchymal stem cells obtained by the method of claim 18.

26. A cryopreserved human mesenchymal stem cells prepared by the stem cells of claims 9, 17 and 25.

27. A cultured system for supporting growth, maintaining undifferentiated state or enhancing differentiation capability of human mesenchymal stem cells, comprising:
a substrate covered with extracellular matrix isolated from human fibroblasts; and
an isolated human mesenchymal stem cells;
wherein the cultured stem cells having at least one of the following characteristics:
a. at least 76% of the cultured stem cells remain undifferentiated for at least 8 or 9 passages; and
b. positive for at least one of the cell markers selected from the group consisting of CD29, CD44, CD90/Thy-1, CD105, CD166, stro-1, SH2, SH3, SH4 and vimentin, and negative for at least one of the cell markers selected from the group consisting of CD31, CD34 and CD45.

28. The cultured system of claim 27, wherein the isolated stem cells is obtained from cord blood or bone marrow.

29. The cultured system of claim 27, wherein the human fibroblasts are a primarily isolated cells or an immortalized cell line.

30. The cultured system of claim 27, further comprising a fibroblast growth factor added to the cultured medium.

31. The cultured system of claim 27, wherein the extracellular matrix is prepared by culturing human fibroblasts, lysing the fibroblasts with alkali solution, and then washing what remains after lysing.

32. The cultured system of claim 27, wherein the undifferentiated stem cells are multipotent.

33. A pharmaceutical composition comprising a human mesenchymal stem cell of claims 9, 17, 25 and 26.

34. A method of treating a patient comprising administering to the patient a therapeutically effective amount of human mesenchymal stem cells of claims 9, 17, 25 and 26.
